(19) United States
(12) Patent Application Publication
(10) Pub. No.: US 2022/0062848 A1
(43) Pub. Date: Mar. 3, 2022

(54) Protein Shells with Nucleic Acid Scaffolds for Use in Biosynthetic Synthesis Pathways

(71) Applicant: Collin Frank Marino, Charlottesville, VA (US)

(72) Inventor: Collin Frank Marino, Charlottesville, VA (US)

(21) Appl. No.: 17/411,575

(22) Filed: Aug. 25, 2021

Related U.S. Application Data
(60) Provisional application No. 63/070,233, filed on Aug. 25, 2020.

(51) Int. Cl. B01J 19/00; C07K 14/195

(52) U.S. Cl. CPC B01J 19/0046; B82Y 30/00; C07K 14/195

(57) ABSTRACT

A nanostructure is provided having a protein shell comprising one or more proteins, at least one nucleic acid scaffold with a plurality of nucleic acid recognition sequences configured to bind to a plurality of enzymes and a plurality of nucleic acid spacers between the plurality of nucleic acid recognition sequences, a linkage between the at least one nucleic acid scaffold and the protein shell, and the plurality of enzymes that are at least partially complementary to the at least one nucleic acid scaffold, each enzyme comprising a nucleic acid binding domain configured to bind the enzyme and the at least one nucleic acid scaffold with some degree of molecular complementarity.

### CROSS-REFERENCE TO RELATED APPLICATIONS

This disclosure claims the benefit, under 35 U.S.C. § 119(e), of U.S. Provisional Patent Application No. 63/070,233, the contents of which are incorporated herein by reference in their entirety

### TECHNICAL FIELD

The present invention provides a versatile and reliable way to improve metabolic flux channeling, pathway orthogonality, production rates, pathway efficiency, and intermediate isolation within synthetic biosynthesis pathways, including by providing multi-protein shells in which nucleic acids are used to orient biosynthesis pathway enzymes.

### BACKGROUND

The use of synthetic biology for the production of various pharmaceuticals, reagents, and other organic compounds has shown great promise over the past decade. However, a significant impediment of this technology is the lack of a versatile and reliable way to improve metabolic flux channeling, pathway orthogonality, production rates, pathway efficiency, and intermediate isolation.

Two previous approaches taken toward this issue include the use of empty BMCs (bacterial microcompartments) in the presence of protein scaffolds to house pathway enzymes in protein shells, and the use of DNA and RNA scaffolds in the absence of any BMCs or protein shells to relatively position pathway enzymes. However, neither of these approaches adequately solve the previously stated issues in a versatile and reliable manner. While the use of DNA and RNA scaffolds have been shown to improve the efficiency of various enzymatic pathways, they fail to isolate the pathways from the cell and are incapable of increasing pathway orthogonality, and isolating intermediates from the chassis organism. The use of BMCs, to which pathway enzymes are individually localized, has also been proposed, but lacks ways to control the relative positions and concentrations of the enzymes and as such does not adequately help to channel metabolic flux.

The creation of chains of proteins has also been proposed to help with flux channeling, and also protein scaffold chains to which other enzymes can be attached. However, these protein-based scaffolds can suffer from complicated secondary and tertiary structures formed by unpredicted interactions between various residues on the scaffold. Furthermore, the use of protein scaffolds inside BMCs has only been theorized, and the solution seems to lack the versatility nucleic acid scaffolds can provide. As an example, linear protein chains are prone to form undesired and virtually unpredictable complexes with themselves, and other proteins, causing the protein scaffolds to lack modularity.

Additionally, an astronomical number of nucleic acid recognition sequences, and nucleic acid binding domains have been identified and created, while the number of non-covalent, commonly used protein-protein linkages is much smaller. This also decreases the modularity of the protein scaffold-based approach. In BMC-free systems, it has also been documented that making modifications to protein-based scaffolds can cause dramatic differences in pathway yields, which once again makes these scaffolds a poor choice for a versatile solution.

To overcome these and other problems, a system is disclosed having multi-protein shells in which stand-alone nucleic acids are used to orient the pathway enzymes relative to each other and the protein shell. This design differs drastically from previous nucleic acid-based scaffolding, in that the scaffolds are “stand-alone”, to distinguish these scaffolds from the plasmid-DNA or genomic nucleic acid scaffolds that have been previously utilized. This distinction allows for the concentration of the scaffolds to be controlled which helps add to the versatility of the design.

This design provides an advantage over the current BMC-based designs by allowing for the versatile control of pathway enzymes relative to a protein shell. The utilization of the nucleic acid scaffolds provides the same benefits as the previously proposed protein scaffolds, but in the absence of the issues caused by the protein scaffolds. The use of nucleic acids also allows for nucleic acid origami (such as DNA origami) based on the scaffolds to be used, thereby furthering the versatility of the nucleic acid scaffold-based design. This novel combination of two previously independent concepts of BMC utilization and nucleic acid scaffolding solves each individual technologies' aforementioned issues.

A system having multi-protein shells in which nucleic acids are used to orient the pathway enzymes is therefore described herein. Pathway enzymes are bound to nucleic acid scaffolds through nucleic acid-binding domains. These scaffolds are bound to the protein shells through either the direct addition of nucleic acid-binding domains to the shell proteins or through the attachment of shell-binding proteins (which interact with the shell through protein-protein binding) to the nucleic acid scaffold via additional nucleic acid-binding domains. The use of multiple different palindromic and/or non-palindromic recognition sequences—to which the nucleic acid-binding domains attach—allows for the specific orientation of pathway enzymes relative to each other and to the protein shell.

### SUMMARY

In accordance with the above, the present invention relates to a system that helps promote diversity and inclusion by recommending people to groups and products/services in order to capitalize on society's diverse makeup.

In one aspect of the invention, a nanostructure is provided having a protein shell having one or more proteins, at least one nucleic acid scaffold with a plurality of nucleic acid recognition sequences configured to bind to a plurality of enzymes and a plurality of nucleic acid spacers between the plurality of nucleic acid recognition sequences, a linkage between the at least one nucleic acid scaffold and the protein shell, and the plurality of enzymes that are at least partially complementary to the at least one nucleic acid scaffold, each enzyme having a nucleic acid binding domain configured to bind the enzyme and the at least one nucleic acid scaffold with some degree of molecular complementarity.

In another aspect of the invention, a process of using a nanostructure is provided, including providing a precursor outside of the nanostructure, diffusing the precursor through the protein shell to a first one of the plurality of enzymes, catalyzed converting, by the first one of the plurality of enzymes, the precursor to a first intermediate molecule, diffusing the first intermediate molecule to a second one of the plurality of enzymes, catalyzed converting, by the second one of the plurality of enzymes, the first intermediate molecule to a desired molecule, and diffusing the desired molecule through the protein shell to the outside of the nanostructure.

### BRIEF DESCRIPTION OF THE DRAWINGS

FIG. 1 shows a schematic in which the nucleic acid scaffolds are directly attached to the protein shell.

FIG. 2 shows a schematic in which the nucleic acid scaffolds are attached to the protein shell via a shell protein binding, nucleic acid domain fusion protein.

FIG. 3 shows operation in a typical application, in which one pathway precursor is converted to a desired product molecule by several intermediate enzymes, and a single coenzyme is recycled by pathway enzymes on an adjacent nucleic acid scaffold.

### DETAILED DESCRIPTION

Described herein is a system having multi-protein shells in which nucleic acids are used to orient the pathway enzymes relative to each other and to the protein shell. The novel use of nucleic acids to attach pathway enzymes to such protein shells is advantageous, as it allows for direct control of enzyme ratios and concentrations relative to the protein shell, spatial orientation of the enzymes relative to the shell, and isolation of pathway components from the external environment by the protein shell.

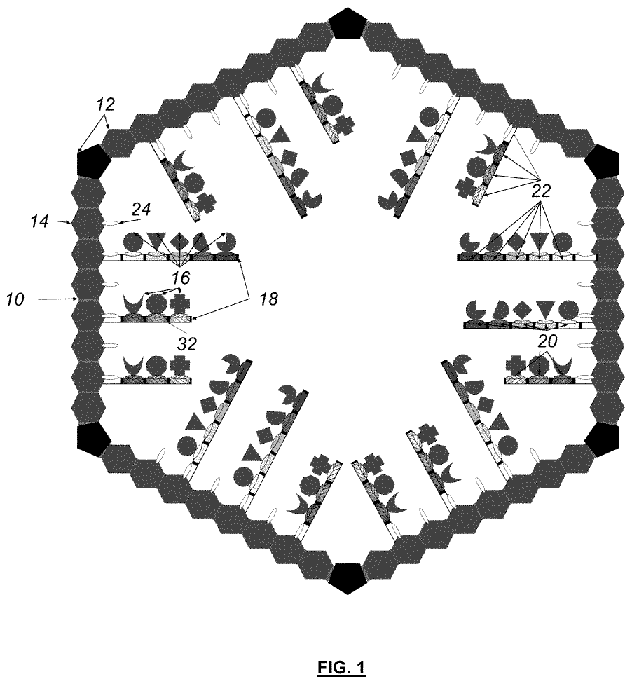

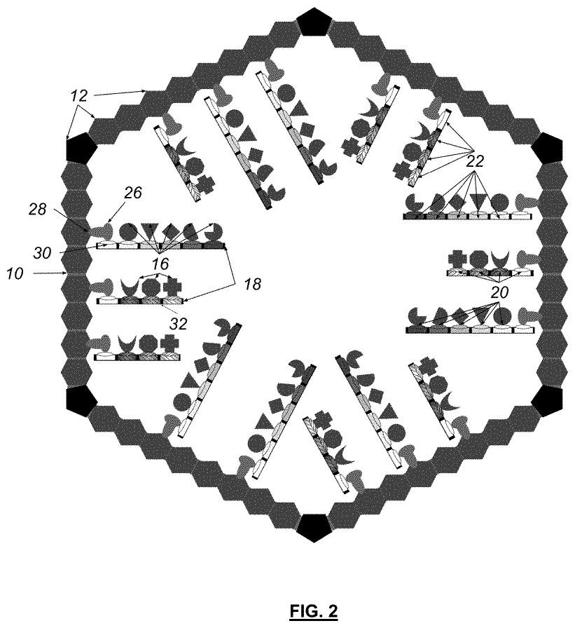

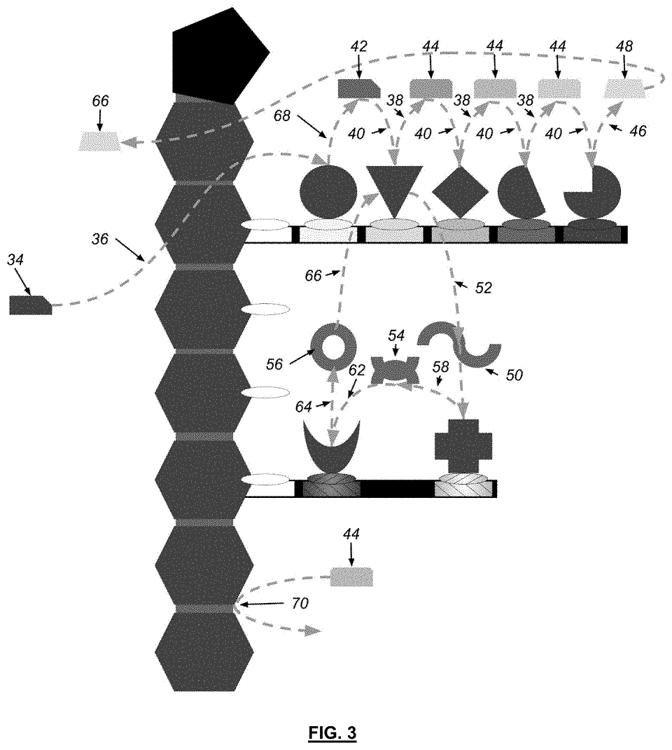

As shown in FIGS. 1-3, a protein shell having one or more proteins, one or more nucleic acid scaffolds of which there can be multiple copies, anabolic and/or catabolic enzymes specific to the desired biosynthesis pathway each containing a nucleic acid binding domain, recognition sequences for the utilized nucleic acid binding domains, nucleic acid spacers, and a linkage between the nucleic acid scaffolds and the protein shell. The protein shell 10 can take the form of any closed or open surface that comprises one or more repeating protein units 12. Examples of valid shells include bacterial microcompartments such as the Pdu, Eut, and carboxysome microcompartments, as well as modified, but not necessarily closed, surfaces composed of mutated versions of these microcompartment shell proteins.

The nucleic acid scaffolds 18 comprise multiple recognition sequences 22 and spacers 32 and can be made from any form of nucleic acid, including: deoxyribonucleic acid, ribonucleic acid, and synthetic nucleic acids such as xeno nucleic acids and peptide nucleic acids among others. These scaffolds are attached to the protein shell. The pathway enzymes are biological proteins whose exact sequences are dependent on the given use case, but which all contain a nucleic acid binding domain either internal to their structure, or at their N or C terminus. Additionally, protein linkers are usually present between this nucleic acid binding domain and the enzyme structure to prevent inhibition of enzyme activity. However, the exact linker(s) used, if any, is(are) also dependent on the specific use case. These pathway enzymes are attached to the nucleic acid scaffolds via their nucleic acid binding domains.

The nucleic acid recognition sequences 22 are unique or semi-unique sequences of nucleic acid monomers on the nucleic acid scaffolds to which the utilized nucleic acid binding domains have some degree of molecular complementarity. These nucleic acid recognition sequences comprise most of the scaffold and mark the locations to which the DNA binding domains of the pathway enzymes attach to the scaffolds. The nucleic acid spacers 32 are relatively short sequences of nucleic acid monomers that are also present on the nucleic acid scaffolds, between the recognition sequences. The linkage between the nucleic acid scaffolds 18 and protein shell 10 provides a method by which the nucleic acid scaffolds are bound to the protein shell through direct or multi-molecule complementarity. This linkage is found between the nucleic acid scaffolds and the protein shell.

One example is through the addition of a nucleic acid binding domain 24 to one or more of the shell proteins forming a nucleic acid binding domain, shell protein fusion 14. Like the pathway enzymes, this nucleic acid binding-domain can be either internal to the shell protein structure or at its N or C terminus, where the exact placement depends on the shell protein being utilized. Alternatively, one or more intermediate proteins can be used to adhere the nucleic acid scaffolds to the shell, where the region of the protein interacting with the shell binds the shell via protein-protein complementarity 28 with a given shell protein, and the region of the protein interacting with the nucleic acid scaffold binds another recognition sequence on the nucleic acid scaffold through another nucleic acid binding domain 30. This forms a shell protein binding, nucleic acid domain fusion 26.

The typical operation consists of eight steps:
a. the diffusion of the pathway precursor(s) through the protein shell,
b. the pathway enzyme catalyzed conversion of the precursor to an intermediate molecule,
c. the migration of this precursor molecule to a neighboring pathway enzyme,
d. the pathway enzyme catalyzed conversion of the nth intermediate molecule to the n+1th intermediate molecule by a neighboring enzyme,
e. the parallel recycling of enzymatic cofactors by adjacent scaffolds containing their own pathway enzymes,
f. the production of the desired output molecule by the last intermediate in the pathway,
g. the diffusion of the output molecule through the protein shell,
h. and inhibition of the diffusion of pathway intermediates.

The protein shell can be made semi-permeable to the pathway precursor(s) 34, allowing for the precursor to diffuse across the protein shell 36. Upon diffusion through the shell, the first enzyme in the pathway can catalyze the production 68 of the first pathway intermediate 42 from the precursor.

This intermediate can then migrate from the first pathway enzyme to the second, via diffusion 40. The distance by which the intermediate needs to diffuse is determined by the nucleic acid spacer placed between the recognition sequences to which the nucleic acid binding domains of the first and second pathway enzymes bind. This helps to improve yields by increasing the probability that the intermediate reaches the next enzyme in the pathway. A similar process then occurs multiple times, as the nth intermediate in the pathway 44 is converted 38 into the n+1th intermediate by a neighboring enzyme. Once again, the distance by which the intermediate needs to diffuse is determined by the nucleic acid spacer placed between the recognition sequences to which the nucleic acid binding domains of the nth and n+1th pathway enzymes bind.

While this is occurring, the coenzyme(s) 56 required by the pathway get recycled by enzymes on adjacent nucleic acid scaffolds. In this process, the used version of the coenzyme 50 is converted 58 into an intermediate version of the coenzyme 54. This intermediate then migrates 62 to the next enzyme on the coenzyme recycling nucleic acid scaffold, where it can be converted 64 to the functioning version of the coenzyme needed by the pathway 56. Additionally, multiple intermediates may be present in the coenzyme recycling reactions, in which case more enzymes would be present on the coenzyme recycling scaffold.

Furthermore, if more than one coenzyme is needed by the pathway, the enzymes required to recycle these coenzymes can be placed on additional, adjacent scaffolds or on the same coenzyme recycling scaffold depending on the application. The last intermediate in the pathway is converted 46 to the desired product molecule 48 by the last enzyme on the scaffold. This product molecule then diffuses through the protein shell.

Finally, intermediates in the pathway are prevented from diffusing across the protein shell 70 by its structure.

Other variations of the foregoing are also desired and taught, including the following:
a. A configuration in which multiple different output molecules are provided can be achieved by tuning the permeability of the shell, as can the positioning of the required enzymes on the scaffold(s).
b. A configuration in which no coenzyme recycling is required for the desired pathway.
c. A configuration in which all pathway enzymes, including those required for coenzyme recycling, are placed on the same scaffold.
d. A configuration in which the final product is sequestered by the protein shell and not allowed to diffuse through it.
e. A configuration in which multiple repeats of the pathway enzymes are placed on the same scaffold.
f. A configuration in which coenzymes and or coenzyme intermediates are allowed to diffuse across the protein shell, and in which coenzyme recycling is performed either entirely external to, or partially external to, the system and not on the scaffolds.
g. As would be understood, any of the foregoing can be combined.

A specific example utilizes a pdu BMC as a protein shell, with the proteins PduA, PduB, PduJ, PduK, PduN, PduU and PduT. Two double stranded DNA scaffolds for the production of resveratrol and the recycling of Coenzyme A respectively. The fusion proteins (4-coumarate-CoA ligase)-(Zif268) and (Stilbene Synthase)-(PBSII) for resveratrol production, and (Acetyl-coenzyme A synthetase 1)-(ZFa) along with an (Acetyl-CoA carboxylase)-(ZFb) for Coenzyme A recycling. The following recognition sequences (written from 5′ to 3′) to which Zif268, PBSII, ZFa, ZFb, and ZFc bind respectively: GCGTGGGCG, GTGTGGAAA, GTCGATGCC, GCGGCTGGG, GAGGACGGC. Nucleic acid spacers of two nucleotides in length. And a linker having a PduD-ZFc fusion protein, and a linker having a PduA-ZFc fusion protein (where these two linkers are being used independently of one another to test two variants of the system). The first of the two scaffold DNAs consists of a ZFc recognition sequence, followed by 4 repeats of a sequence containing a two nucleotide spacer followed by a Zif268 recognition sequence, followed by a two nucleotide spacer, followed by a PBSII recognition sequence. The second of the two scaffold DNAs consists of a ZFc recognition sequence, followed by 4 repeats of a sequence containing a two nucleotide spacer followed by a ZFa recognition sequence, followed by a two nucleotide spacer, followed by a ZFb recognition sequence. This system is being assembled inside of a 10-beta Competent E. coli by three parts:
a. a part encoding the (4-coumarate-CoA ligase)-(Zif268), (Stilbene Synthase)-(PBSII), (Acetyl-coenzyme A synthetase 1)-(ZFa) and (Acetyl-CoA carboxylase)-(ZFb) fusion proteins,
b. a part encoding the PduA, PduB, PduJ, PduK, PduN, PduU, PduT and PduD-ZFc/PduA-ZFc proteins,
c. and a part encoding r_oligo genes corresponding to both DNA scaffold sequences in addition to their reverse compliments along with the enzymes Human Immunodeficiency Virus Reverse Transcriptase, and Murine Leukemia Virus Reverse Transcriptase.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect.

For purposes of explanation and not limitation, specific details are set forth such as particular architectures, interfaces, techniques, etc. to provide a thorough understanding. In other instances, detailed descriptions of well-known devices, circuits, and methods are omitted so as not to obscure the description with unnecessary detail.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc. for different features/components of the present disclosure are only intended to distinguish the features/components from other similar features/components and not to impart any order or hierarchy to the features/components.

To aid the Patent Office and any readers of any patent issued on this application in interpreting the claims appended hereto, Applicant that it does not intend any of the claims or claim elements to invoke 35 U.S.C. 112(f) unless the words “means for” or “step for” are explicitly used in the particular claim.

While the present teachings have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings.

What is claimed is:

1. A nanostructure comprising:
(a) a protein shell having one or more proteins;
(b) at least one nucleic acid scaffold having:
(i) a plurality of nucleic acid recognition sequences configured to bind to a plurality of enzymes, and
(ii) a plurality of nucleic acid spacers between the plurality of nucleic acid recognition sequences;
(c) a linkage between the at least one nucleic acid scaffold and the protein shell; and
(d) the plurality of enzymes that are at least partially complementary to the at least one nucleic acid scaffold, each enzyme having a nucleic acid binding domain configured to bind the enzyme and the at least one nucleic acid scaffold with some degree of molecular complementarity.

2. The nanostructure of claim 1, wherein the protein shell comprises a bacterial microcompartment or a modified version thereof.

3. The nanostructure of claim 1, wherein each of the plurality of the nucleic acid recognition sequences comprises unique or semi-unique sequences of nucleic acid monomers.

4. The nanostructure of claim 1, wherein each of the plurality of nucleic acid spacers comprises relatively short sequences of nucleic acid monomers.

5. The nanostructure of claim 1, wherein the linkage between the at least one nucleic acid scaffold and the protein shell comprises another nucleic acid binding domain which is added to the one or more proteins, and wherein the another nucleic acid binding domain is internal to the one or more proteins or external at N or C terminus of the one or more proteins.

6. The nanostructure of claim 1, wherein the linkage between the at least one nucleic acid scaffold and the protein shell comprises an intermediate protein which binds to the protein shell with protein-protein complementarity and binds to the at least one nucleic acid scaffold with the intermediate protein's nucleic acid binding domain.

7. The nanostructure of claim 1, wherein the plurality of enzymes comprise anabolic or catabolic enzymes which are biological proteins whose sequences are dependent on a given use for desired biosynthesis pathway.

8. The nanostructure of claim 1, wherein the nucleic acid binding domain of each enzyme is internal to the enzyme or external at N or C terminus of the enzyme.

9. The nanostructure of claim 1, further comprising a protein linker between each enzyme and the nucleic acid binding domain of the enzyme, the protein linker configured to prevent inhibition of enzyme activity.

10. The nanostructure of claim 1 is used for production of a material when a precursor is introduced to the nanostructure.

11. A process of using the nanostructure of claim 1, comprising:
providing a precursor outside of the nanostructure;
diffusing the precursor through the protein shell to a first one of the plurality of enzymes;
catalyzed converting, by the first one of the plurality of enzymes, the precursor to a first intermediate molecule;
diffusing the first intermediate molecule to a second one of the plurality of enzymes;
catalyzed converting, by the second one of the plurality of enzymes, the first intermediate molecule to a desired molecule; and
diffusing the desired molecule through the protein shell to the outside of the nanostructure.

12. The process of claim 11, further comprising recycling a coenzyme required by the process by using two adjacent enzymes which are attached on a coenzyme recycling nucleic acid scaffold.

13. The process of claim 12, wherein recycling the coenzyme further comprises: converting, by one of the two adjacent enzymes, a used version of the coenzyme to an intermediate version of the coenzyme; diffusing the intermediate version of the coenzyme from the one of the two adjacent enzymes to another of the two adjacent enzymes; and converting, by the another of the two adjacent enzymes, the intermediate version of the coenzyme to a usable version of the coenzyme.

14. The process of claim 12, wherein the coenzyme recycling nucleic acid scaffold is same as the at least one nucleic acid scaffold.

15. The process of claim 11, wherein the protein shell is pdu BMC, and the at least one nucleic acid scaffold is double stranded DNA scaffold.

16. The process of claim 11, wherein the precursor is 4-coumaric acid, and the desired molecule is resveratrol.

17. The process of claim 11, wherein the plurality of enzymes sequentially convert the first intermediate molecule into a plurality of additional intermediate molecules including a final intermediate molecule, and the final intermediate molecule is converted to the desired molecule by a final one of the plurality of enzymes.

18. The process of claim 17, wherein at least one of the plurality of the intermediate molecule is prevented from diffusing through the protein shell to the outside of the nanostructure.

19. The process of claim 11, further comprising recycling a coenzyme required by the process by using a plurality of recycling enzymes which are attached on a coenzyme recycling nucleic acid scaffold.

20. The process of claim 19, wherein recycling the coenzyme further comprises: sequentially converting, by the plurality of recycling enzymes, a used version of the coenzyme to a plurality of intermediate version of the coenzyme including a final intermediate version of the coenzyme, wherein the final intermediate version of the coenzyme is converted to a usable version of the coenzyme by a final one of the plurality of recycling enzymes.
